(19) United States
(12) Patent Application Publication
(10) Pub. No.: US 2019/0388276 A1
(43) Pub. Date: Dec. 26, 2019

(54) SNOW GOGGLE STRUCTURE

(71) Applicant: SUN SIGHT GLASSES CO., LTD., Tainan (TW)

(72) Inventor: JUI-CHI LI, TAINAN (TW)

(21) Appl. No.: 16/283,990

(22) Filed: Feb. 25, 2019

(30) Foreign Application Priority Data
Jun. 26, 2018 (TW) 107208556

(51) Int. Cl. A61F 9/02

(52) U.S. Cl. CPC A61F 9/025; A61F 9/028

(57) ABSTRACT

A snow goggle structure is disclosed herein. It comprises a main frame having an assembly hole defined by an upper frame, a left frame, a lower frame and a right frame, and at least one first magnetic element disposed on each of the left frame, the lower frame and the right frame, wherein the upper frame is provided with a first fastener; and a lens assembly disposed on an outer side of the main frame and having a lens frame, a lens assembled to the lens frame and at least one second magnetic element disposed on the lens frame for correspondingly attracting the at least one first magnetic element of the main frame, wherein the lens frame is provided with a second fastener for correspondingly fastening to the first fastener of the main frame.

### BACKGROUND OF THE INVENTION

### 1. Field of the Invention

The present invention relates to a snow goggle structure which comprises the lens to be replaced quickly and easily and improves heat dissipation efficiency.

### 2. Description of Related Art

When people are doing outdoor activities in the snow, their vision may be affected by the light reflected by the snow on the ground, the cold wind and the low temperature. Accordingly, people usually wear snow goggles to protect the eyes from being affected or damaged by wind, snow, or other intrusive objects and to ensure that they can clearly see the surrounding environment during outdoor activities.

Generally, under different environmental conditions, e.g. high or low light intensity environment, people choose snow goggles having different visible light transmission lenses to wear. In order to effectively resist the cold wind, it is necessary to wear the snow goggles on the face near the wearer's eyes during use. However, in extremely low temperature environments, the hot air emanating from the human skin and exhaled from the nasal cavity accumulates as fogs on the lenses of the snow goggles, so that people cannot clearly see the surrounding environment. Therefore, hot air must be effectively removed to avoid fogs formation on the lenses of the snow goggles.

### SUMMARY OF THE INVENTION

Disclosed herein is a snow goggle structure. It comprises a main frame and a lens assembly disposed on an outer side of the main frame. The main frame has an assembly hole defined by an upper frame, a left frame, a lower frame and a right frame, and at least one first magnetic element disposed on each of the left frame, the lower frame and the right frame. The upper frame is further provided with a first fastener. The lens assembly has a lens frame, a lens assembled to the lens frame and at least one second magnetic element disposed on the lens frame for correspondingly attracting the at least one first magnetic element of the main frame. The lens frame is provided with a second fastener for correspondingly fastening to the first fastener of the main frame.

According to an embodiment of the present invention, the first fastener is an engaging slot, and the second fastener is a retractable engaging rod.

According to an embodiment of the present invention, the first fastener is a push latch seat, and the second fastener is a bolt.

According to an embodiment of the present invention, the lower frame of the main frame is provided with a fixing hole, and the lens frame is provided with a positioning part for correspondingly inserting into the fixing hole.

According to an embodiment of the present invention, an inner side of the main frame is provided with an annular wall having at least one ventilation hole.

Accordingly, the main frame and the lens assembly of the present invention are assembled by the fasteners and the magnetic elements, so the lens can be replaced quickly and easily. Furthermore, the present invention improves heat dissipation efficiency in use by releasing the first fastener from the second fastener to form the opening between the main frame and the lens assembly.

### BRIEF DESCRIPTION OF THE DRAWINGS

FIG. 1 is a stereogram showing a snow goggle structure according to the present invention;

FIG. 2 is an exploded view showing a snow goggle structure according to the present invention;

FIG. 3 is a cross-sectional view showing a main frame assembled to a lens assembly according to the present invention;

FIG. 4 is a cross-sectional view showing a snow goggle structure in assembly according to the present invention;

FIG. 5 is a cross-sectional view showing an opening to dissipate hot air according to the present invention.

### DETAILED DESCRIPTION OF THE PREFERRED EMBODIMENT

Hereinafter, an exemplary embodiment of the present invention will be described in detail with reference to the accompanying drawings.

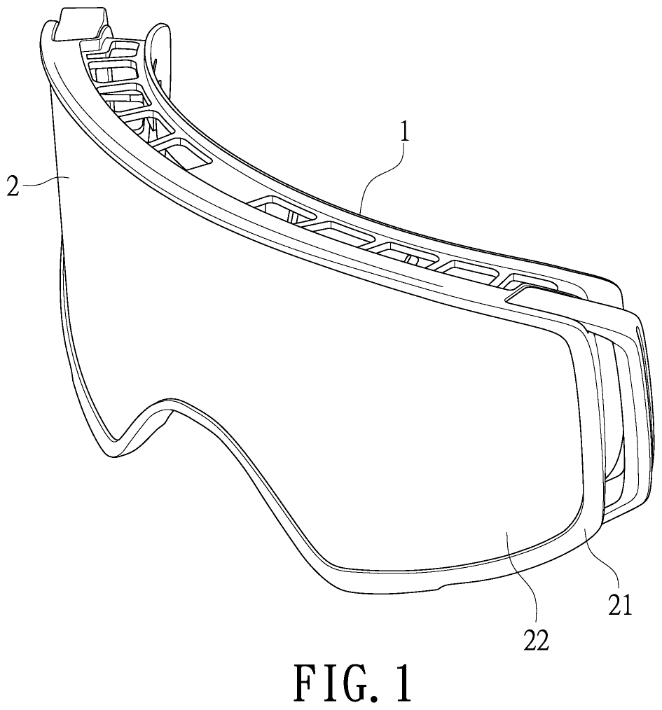

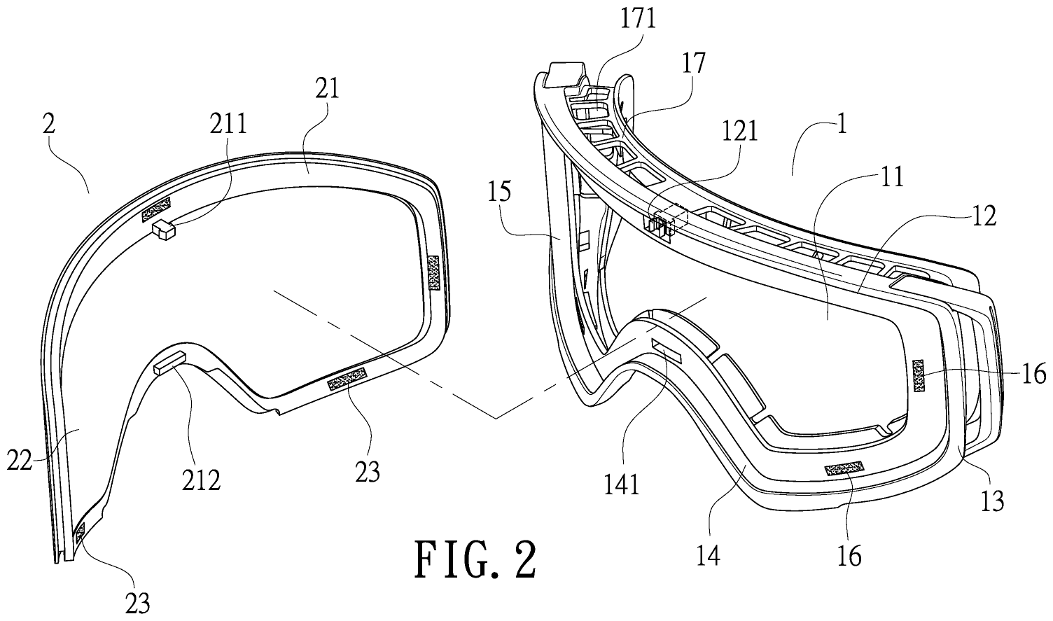

As showed in FIG. 1 and FIG. 2, a stereogram showing and an exploded view showing a snow goggle structure according to the present invention are disclosed.

The snow goggle structure mainly comprises a main frame (1) and a lens assembly (2). The main frame (1) is provided with an assembly hole (11) defined by an upper frame (12), a left frame (13), a lower frame (14) and a right frame (15), and at least one first magnetic element (16) disposed on each of the left frame (13), the lower frame (14) and the right frame (15). The upper frame (12) is provided with a first fastener (121), and the first fastener (121) can be disposed at a center of the upper frame (12). Furthermore, the lower frame (14) is provided with a fixing hole (141).

The lens assembly (2) is disposed on an outer side of the main frame (1) and has a lens frame (21), a lens (22) assembled to the lens frame (21) and at least one second magnetic element (23) disposed on the lens frame (21) for correspondingly attracting the at least one first magnetic element (16) of the main frame (1). The lens frame (21) is provided with a second fastener (211) for correspondingly fastening to the first fastener (121) of the main frame (1) and a positioning part (212) for correspondingly inserting into the fixing hole (141) of the lower frame (14).

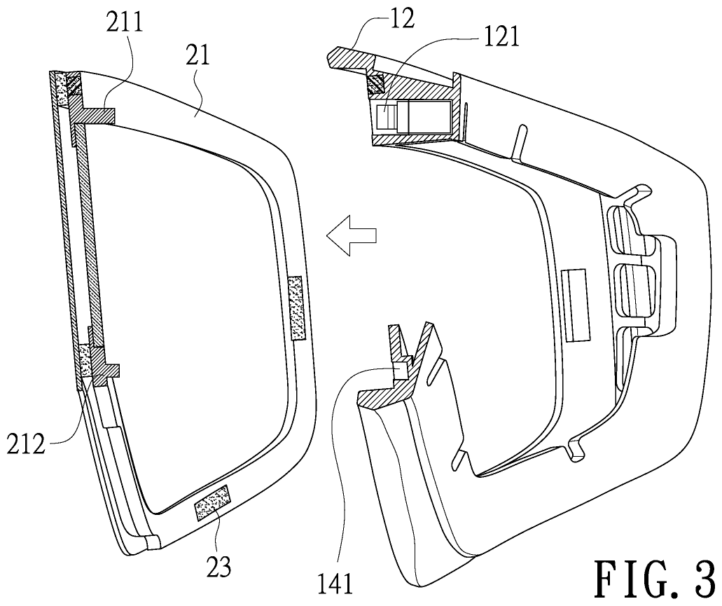

Referring to FIG. 2 and FIG. 3, in assembling the present invention, the positioning part (212) is inserted into the fixing hole (141) to make the at least one first magnetic element (16) on the main frame (1) magnetically attract the at least one second magnetic element (23) on the lens assembly (2). Then, the first fastener (121) on the upper frame (12) is buckled to the second fastener (211) on the lens frame (21), so that the main frame (1) and the lens assembly (2) are assembled. Preferably, the first fastener (121) is an engaging slot seat, and the second fastener (211) is a retractable engaging rod; or the first fastener (121) is a push latch, and the second fastener (211) is a bolt. Therefore, the second fastener (211) can be pushed to buckle the first fastener (121). If the lens (22) is needed to be replaced, the second fastener (211) is first pressed to release the fastening state, and then the at least one first magnetic element (16) is detached from the at least one second magnetic element (23) by applying a force. In such a case, the lens assembly (2) can be removed from the main frame (1) to replace the other lenses, which is very convenient and quick to operate.

Referring to FIG. 2, an inner side of the main frame (1) is provided with an annular wall (17), and the annular wall (17) is provided with at least one ventilation hole (171) to dissipate hot air. When a user wears the snow goggle structure, the inner side of the main frame (1) contacts the skin near the eyes, and the hot air emanating from the skin and exhaled from the nasal cavity is dissipated from the at least one ventilation hole (171), which prevents fogs accumulating on the lens (22).

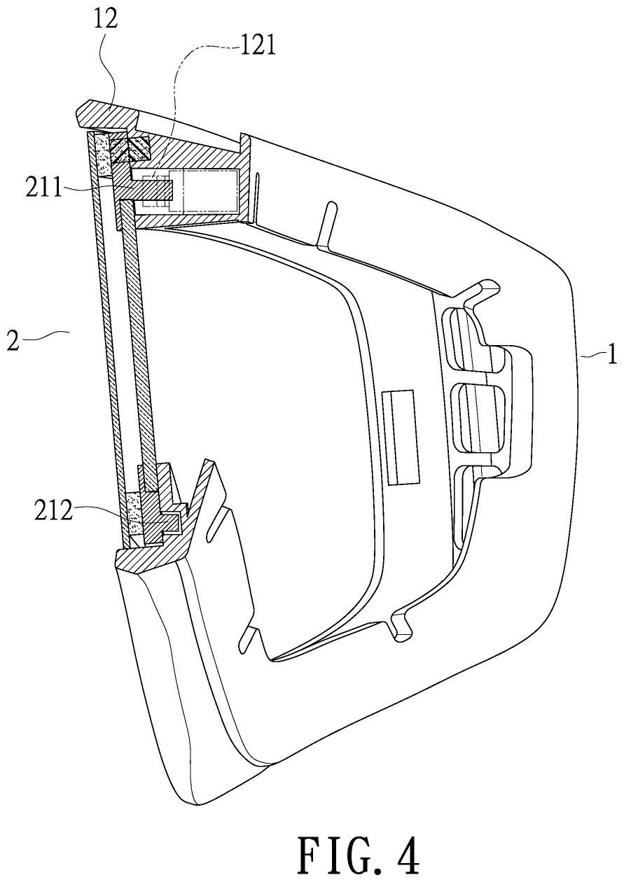

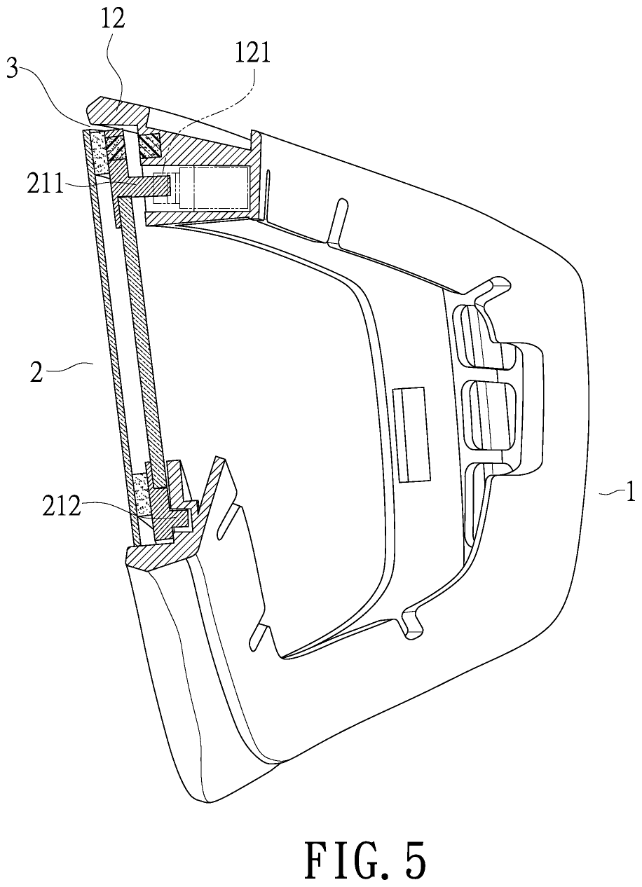

Additionally, referring to FIG. 4 and FIG. 5, the at least one first magnetic element (16) and the at least one second magnetic element (23) are still magnetically attracted to each other even when the engaging state of the first fastener (121) and the second fastener (211) is released. Therefore, the main frame (1) and the lens assembly (2) are not completely separated from each other, and only an opening (3) is formed between the main frame (1) and the lens assembly (2) to accelerate the discharge of the hot air.

Compared with the technique available now, the present invention has the following advantages:

1. The main frame of the invention is assembled to the lens assembly by the magnetic elements and the fasteners, so the present invention has the advantages of simple assembly and easy and fast replacement of the lens.

2. Two different combination ways of the magnetic elements and the fasteners strengthen the combination of the main frame and the lens assembly, so the present invention prevents the lens assembly from being separated from the main frame due to accidental contact or collision during the activity.

3. The invention generates the opening between the lens assembly and the main frame to remove the hot air by releasing the fastening state of the first fastener and the second fastener, so the present invention improves the removal rate of hot air.

What is claimed is:

1. A snow goggle structure, comprising:
a main frame having an assembly hole defined by an upper frame, a left frame, a lower frame and a right frame, and at least one first magnetic element disposed on each of the left frame, the lower frame and the right frame, wherein the upper frame is provided with a first fastener; and
a lens assembly disposed on an outer side of the main frame and having a lens frame, a lens assembled to the lens frame and at least one second magnetic element disposed on the lens frame for correspondingly attracting the at least one first magnetic element of the main frame, wherein the lens frame is provided with a second fastener for correspondingly fastening to the first fastener of the main frame.

2. The snow goggle structure as claimed in claim 1, wherein the first fastener is an engaging slot seat, and the second fastener is a retractable engaging rod.

3. The snow goggle structure as claimed in claim 1, wherein the first fastener is a push latch, and the second fastener is a bolt.

4. The snow goggle structure as claimed in claim 1, wherein the lower frame of the main frame is provided with a fixing hole, and the lens frame is provided with a positioning part for correspondingly inserting into the fixing hole.

5. The snow goggle structure as claimed in claim 1, wherein an inner side of the main frame is provided with an annular wall having at least one ventilation hole.
